(19) United States
(12) Patent Application Publication
(10) Pub. No.: US 2005/0090875 A1
(43) Pub. Date: Apr. 28, 2005

(54) Optical projection and tracking system for retinal prosthesis

(76) Inventors: Daniel V. Palanker, Sunnyvale, CA (US); Alexander Vankov, Mountain View, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 10/939,232

(22) Filed: Sep. 9, 2004

Related U.S. Application Data
(60) Provisional application No. 60/501,920, filed on Sep. 10, 2003.

(51) Int. Cl. A61F 9/00

(52) U.S. Cl. 607/54

(57) ABSTRACT

A system having a retinal prosthesis inside a mammalian eye and an external imaging unit outside the eye is provided. The external imaging unit includes an imager which receives an input optical image and a display which provides a processed optical image derived from the input optical image as an input to the eye. The external imaging unit also includes a tracking subsystem, to determine the position of the retinal prosthesis relative to the display. The external imaging unit includes an image processor, which performs spatial processing dependent on the position of the retinal prosthesis relative to the display. Thus the processed image provided to the eye is spatially processed according to the position of the retinal prosthesis. The image processor can perform other kinds of image processing as well (e.g., temporal image processing). An external imaging unit for use in such a system is also provided.

### CROSS REFERENCE TO RELATED APPLICATIONS

This application is related to and claims priority from U.S. provisional application 60/501,920, filed Sep. 10, 2003, entitled “Optical Projection and Tracking System for Retinal Prosthesis”, and hereby incorporated by reference.

### FIELD OF THE INVENTION

This invention relates to vision improvement by use of a retinal prosthesis.

### BACKGROUND

Some common conditions that can lead to partial or total blindness (e.g., retinitis pigmentosa and age-related macular degeneration) result mainly from degeneration of retinal photoreceptors. However, in these conditions, the wiring and processing functionality of the retina is typically preserved to a significant degree. Thus retinal prostheses that provide the lost photoreceptor functionality by stimulating retinal neural cells are being actively investigated as a treatment method for such conditions.

In general terms, the functionality provided by such a system is the selective excitation of retinal neural cells according to an optical image input. Many different approaches have been developed to perform this function. The input optical image is usually acquired with a video camera or other standard imaging device located outside the eye. Image information (and often electrical power) can be transmitted serially to a receiver on a retinal prosthesis located inside the eye with a point to point wireless link (e.g., an RF link or an optical link). In principle, a wire could be also used for such a link, but there are clear practical difficulties with a wired link.

Even for wireless links, there are significant practical issues, such as excessive intraocular power dissipation (especially for a retinal prosthesis having a large number of pixels). In particular, conversion of a serial stream of information to a form suitable for retinal stimulation can significantly increase total intra-ocular power dissipation. Such approaches are considered by Krulevitch et al. in U.S. Patent Applications 2003/0097165 and 2003/0097166; by Ok et al. in U.S. Patent Application 2002/0095193; by Scribner in U.S. Patent Application 2002/0161417; and by Greenberg et al. in U.S. Patent Applications 2002/0010496, 2002/0193845, 2002/0091421 and 2002/0091422.

Another approach for providing input to a retinal prosthesis is to optically project image information onto the retinal prosthesis. Nisch et al. in U.S. Pat. Nos. 6,298,270 and 6,347,250 considers retinal prostheses suitable for use in such a system. Greenberg et al., in U.S. Pat. No. 6,507,758, considers an external unit providing image intensification for such a system. Chow et al., in U.S. Pat. No. 5,895,415, also considers an imaging retinal prosthesis system, where an external unit provides certain kinds of image processing.

However, these image projection retinal prosthesis systems still suffer from certain drawbacks. These prior art approaches do not easily provide a wide field of view that can be observed by a user with natural eye movements. For example, U.S. Pat. No. 5,895,415 considers a mechanically complicated arrangement where eye position is tracked and the input video imaging camera is pointed to a corresponding direction by a mechanical system. It is also not straightforward to use such systems with a retinal prosthesis at or near the macula of the eye, because there is a non-local relation between photoreceptors and corresponding neural cells at the macula.

Accordingly, it would be an advance in the art to provide, in a simple manner, an imaging retinal prosthesis system having a wide field of view. It would also be an advance in the art to provide an imaging retinal prosthesis system capable of simply compensating for a non-local relation between photoreceptors and corresponding retinal neural cells (e.g., at the macula).

### SUMMARY

The present invention provides a system having a retinal prosthesis inside a mammalian eye and an external imaging unit outside the eye. The external imaging unit includes an imager which receives an input optical image and a display which provides a processed optical image as an input to the eye. The external imaging unit also includes a tracking subsystem, to determine the position of the retinal prosthesis relative to the display. The external imaging unit includes an image processor, which performs spatial processing dependent on the position of the retinal prosthesis relative to the display. Thus the processed image provided to the eye is spatially processed according to the position of the retinal prosthesis. The image processor can perform other kinds of image processing as well (e.g., temporal image processing). An external imaging unit for use in such a system is also provided.

The retinal prosthesis of such a system can include reference points to facilitate tracking. The retinal prosthesis can include a power receiver for receiving optical power provided by the external imaging unit display. Tracking the position of the retinal prosthesis allows alignment of delivered power to the optical power receiver.

Some advantages of the present invention include:
1) The external imaging unit can emit as much power as the eye can tolerate (typically about a few mW, limited by retinal heating) thus providing a robust signal to each pixel of the retinal prosthesis.
2) Transmission of information from the external display to each pixel in the retinal prosthesis is performed by transmission of an optical image. Thus there is no need for decoding of a serial transmission, and no intraocular decoding processor is required, in contrast with approaches having a single serial link serving all pixels.
3) The external display can emit in the near-infrared part of the spectrum in order to avoid stimulation of remaining photoreceptors in the eye and to reduce heating of the eye due to absorption of light in blood, in remaining photoreceptors and in pigmented epithelium and other ocular pigments. At the same time, peripheral vision of the patient can be maintained to provide conventional color vision in the areas of an image outside the retinal implant.
4) The external display can project an image onto the retina that is much larger than the retinal prosthesis. Thus the patient can use natural eye movements in order to observe a larger field of view than provided by the retinal prosthesis.
5) The intensity, duration and repetition rate of the stimulating signal produced by the retinal prosthesis can be controlled by the intensity, duration and repetition rate of the external display. These parameters can be adjusted without making any changes in the retinal prosthesis. This feature provides flexibility in optimization of the stimulation parameters, which may have to be adjusted for each patient.
6) The invention is applicable to both epiretinal and subretinal implants.
7) The invention can be practiced with many kinds of retinal prostheses. In particular, retinal prostheses having electrical, mechanical or chemical stimulation can be employed according to the invention.

### BRIEF DESCRIPTION OF THE DRAWINGS

FIG. 1 shows a vision improvement system according to an embodiment of the invention.

FIG. 2a shows a subretinal retinal prosthesis.

FIG. 2b shows an epiretinal retinal prosthesis.

FIG. 3 schematically shows the arrangement of photoreceptors and corresponding retinal neural cells at the foveola.

FIG. 4a shows a photograph of a typical scene.

FIG. 4b shows the scene of FIG. 4a where a part of the scene selected according to retinal prosthesis position is spatially processed.

FIG. 4c shows the scene of FIG. 4b where a different part of the scene is spatially processed (corresponding to a different position of the retinal prosthesis).

### DETAILED DESCRIPTION

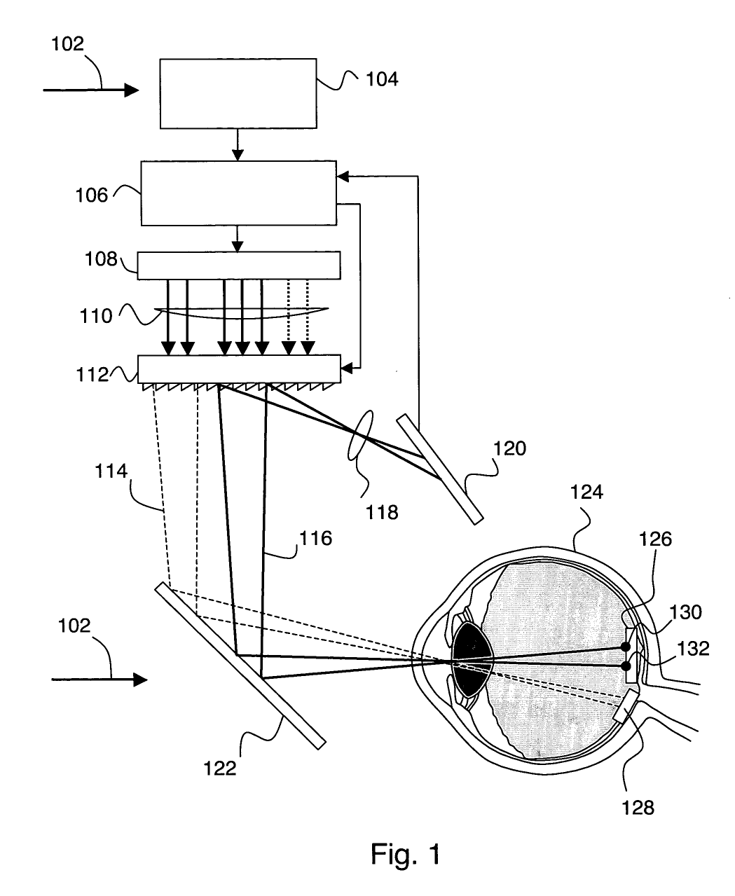

FIG. 1 shows a vision improvement system according to an embodiment of the invention. An input optical image 102 is received by an imager 104. Imager 104 can be a video camera, digital camera or any other standard imaging device providing an output suitable for image processing. Imager 104 provides an input to an image processor 106, which can be any combination of hardware and/or software suitable for performing the functions described herein. Image processor 106 performs image processing on input optical image 102 and provides a processed image derived from this input optical image. Further details and examples of such image processing will be given below.

A display driven by image processor 106 provides the processed image as an optical input to an eye 124 having a retinal prosthesis 126. In the preferred embodiment of FIG. 1, the display includes a source array 108 of collimated infrared light emitting diodes (LEDs), a condensing lens 110, and a liquid crystal display (LCD) 112. More generally, the display can be any device capable of providing the processed image as an optical input to eye 124. Preferably, the display has a field of view significantly larger than the field of view of retinal prosthesis 126. With such a display, a large field of view (e.g., a field of view comparable to that of eye 124) can be observed by a user with natural eye movements. Note that this large field of view is provided without requiring mechanical motion of imager 104. The preferred display shown on FIG. 1 has several features which reduce power consumption and thereby provide a more efficient system.

Preferably source array 108 and/or LCD 112 are selectively excited, such that only the elements emitting light that is projected on or near the retinal prosthesis (solid arrows) are energized, while elements emitting light that is projected away from the retinal prosthesis (dotted arrows) are not energized. In this manner, the power delivered to eye 124 (i.e., the heat load) from source array 108 through LCD 112 can be significantly reduced. This, in turn, allows use of brighter LEDs in source array 108 which reduces the level of electrical amplification needed in each pixel of the retinal prosthesis. The image displayed by LCD 112 onto retinal prosthesis 126 preferably has an intensity suitable for operation of retinal prosthesis 126, which can entail intensification of optical input image 102, especially for low ambient light levels.

In some cases (e.g., as shown on FIG. 1) an intra-ocular power receiver 128 receives light from the display (shown following path 114 on FIG. 1). Optical power delivered to power receiver 128 can be used to provide electrical power to retinal prosthesis 126, e.g., with a wire (not shown). Although prosthesis 126 and receiver 128 are shown as separate entities on FIG. 1, it is also possible for these to be integrated in the same structure (or chip). In cases where a power receiver is employed, it is preferred to include the elements of source array 108 and/or LCD 112 that emit/transmit light projected onto power receiver 128 in the set of source array elements to be selectively excited. Projection at maximum intensity onto power receiver 128 is also preferred, subject to limits set by the need to avoid any damage to eye 124. Note that light projected onto power receiver 128 is preferably projected at constant or near-constant intensity to maximize power transfer, while light projected onto retinal prosthesis 126 may be pulsed to improve neural cell stimulation.

The elements of source array 108 are preferably relatively well collimated (e.g., they have a divergence full angle of about 8 degrees or less), and condensing lens 110 is preferably designed to direct such collimated source radiation toward the center of eye 124. These two features significantly improve system efficiency relative to a system where source array 108 emits radiation equally in all directions, since a small fraction (typically <1%) of such isotropic radiation is collected by eye 124, and a small fraction (about 5%) of light collected by eye 124 reaches prosthesis 126. For example, to provide about 1.5 mW of light to a 3 mm diameter retinal prosthesis, the preferred arrangement shown on FIG. 1 requires only about 5 mW of optical power from the display. A large field of view display having isotropic and non-selective backlighting could require as much as 7.5 W of optical power to provide the same illumination to the retinal prosthesis.

Light from LCD 112 traveling toward eye 124 along optical path 116 is projected onto retinal prosthesis 126. In this manner, optical input is provided to retinal prosthesis 126, which in turn stimulates neural cells in the retina of eye 124. Optionally, as indicted above, light from the display traveling toward eye 124 along path 114 is projected onto power receiver 128. In this manner, electrical power can be provided to retinal prosthesis 126. For example, a connection (not shown) between power receiver 128 and retinal prosthesis 126 can provide such electrical power.

Optical paths 114 and 116 are shown reflected from an IR-reflective screen 122. Screen 122 is preferably also transmissive to visible light, such that visible optical input 102 can pass through screen 122 and enter eye 124. Such visual input to eye 124 can allow coordination of remaining normal color peripheral vision with vision provided by the retinal prosthesis system. To facilitate such coordination, it is preferred for source array 108 to emit radiation at a non-visible wavelength (e.g., at a near infrared wavelength). Selection of such a non-visible wavelength also reduces intra-ocular heating due to absorption of light from source array 108. It is also preferred for retinal prosthesis 126 to be sensitive to the wavelength emitted by source array 108 and less sensitive to visible light.

In order to accomplish many of the above functions, it is necessary to determine the position of retinal prosthesis 126 relative to LCD 112 (or more generally, relative to the external display). Accordingly, the system of FIG. 1 includes a tracking subsystem for determining this position. In the preferred embodiment of FIG. 1, tracking is facilitated by provision of several reference points 130 and 132 on retinal prosthesis 126. Although two reference points are sufficient for determining retinal prosthesis position, the use of more than two reference points can increase tracking accuracy and/or stability. Reference points 130 and 132 can be passive devices responsive to incident light (e.g., a corner cube retro-reflector or a small piece of fluorescent material), or can be active devices such as light emitting diodes. It is preferable for the radiation emitted by the reference points to differ from the radiation supplied by source array 108 (e.g., in wavelength or in time-dependence), to facilitate distinguishing radiation from the reference points from other radiation in the system.

Methods for distinguishing between the reference points and the rest of the retinal prosthesis include the following: 1) The reference points can have enhanced reflectivity and/or scattering compared to the rest of the retinal prosthesis surface. It is important to return the reflected or scattered light back through the pupil of the eye in order to be able to detect this light. To assure the reflection of the light back into the pupil for any orientation of the retinal prosthesis, the reference point can be made as a 3-mirror back-reflector (also known as a corner cube reflector). 2) To improve contrast between light emitted by the reference points and light scattered from other places in the eye, the reference points can be made fluorescent, and the imaging of the reference points on the tracking array can be performed at the wavelength of fluorescence, rather than at the illumination wavelength. 3) Reference points can be light emitting diodes (LED) or other active light emitters at a wavelength different from that used by the display. Detection of light at the reference point wavelength by the tracking array will reduce the background signal produced by the scattered light from the eye. The light emitted by the reference points can also be distinguished from the scattering if it has a different time-dependence. For example, reference point emitters can be detected at times when the display is not providing any light to eye 124.

Light from reference points 130 and 132 travels from eye 124 toward LCD 112, and is deflected onto a tracking array 120. Tracking array 120 can be an array of photodiodes, or can be any other device suitable for providing position information. Tracking array 120 is preferably disposed in a plane conjugate to the plane of LCD 112 (e.g., as determined by an imaging lens 118). Tracking array 120 provides input to image processor 106. Light from eye 124 can be deflected onto tracking array 120 by a beam splitter (not shown), but the more compact arrangement shown on FIG. 1 where angled faces on the surface of LCD 112 deflect some light toward tracking array 120 is preferred.

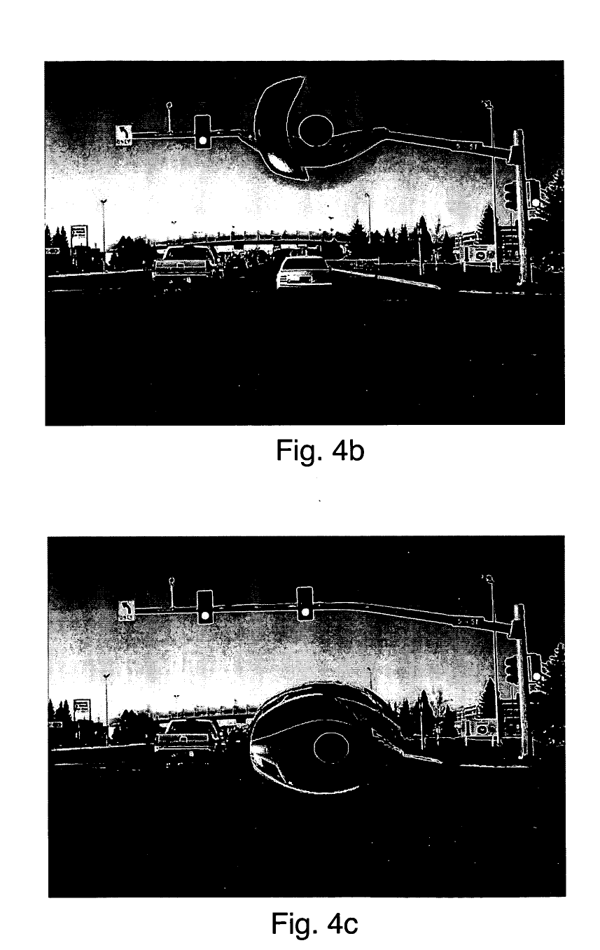

Therefore, the image processing provided by image processor 106 includes spatial image processing dependent on the position of the retinal prosthesis relative to the display. Such position-dependent spatial image processing is a key feature of the present invention. The above-described selective excitation of source array elements for providing image light (and optionally power light) to eye 124 is an example of such position-dependent spatial processing. More specifically, this is a 2-D cropping function, where parts of the processed image that would not be projected onto retinal prosthesis 126 are not displayed. For reasons of efficiency, it is preferable to implement this cropping function by selective excitation of the source array 108 (as opposed to the alternative of cropping with LCD 112). Such position-dependent spatial processing can include image intensification, 1-D or 2-D rectangular scaling, 2-D radial scaling, rectangular or radial cropping, rotation and/or translation. Further examples of such position-dependent spatial processing are considered in connection with FIGS. 4a-c.

Elements 104-122 shown on FIG. 1 are preferably included in an external imaging unit for use with retinal prosthesis 126. Such an external imaging unit can be packaged as goggles or glasses for convenient long term use by a patient. Image processor 106 can be integrated with such goggles or glasses, or can be in a separate unit electrically connected to the goggles or glasses, and also worn or carried by the patient (e.g., in a pocket or attached to an article of clothing).

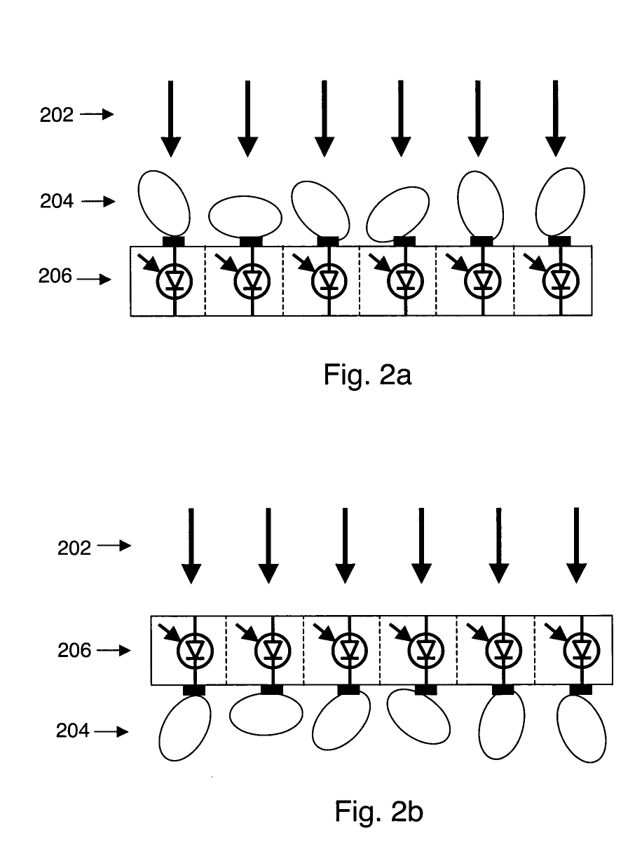

FIGS. 2a and 2b schematically show a retinal prosthesis suitable for use with the present invention. FIGS. 2a and 2b show subretinal and epiretinal positioning of a retinal prosthesis respectively. Optical pixel inputs 202 are incident on an array of photosensitive pixels 206. With reference to FIG. 1, optical pixel inputs 202 can be an image projected from LCD 112, and electrical power for the prostheses of FIGS. 2a and 2b can be provided by power supply 128. Each pixel has an electrode for localized stimulation of retinal neural cells 204. Typically, a retinal prosthesis as on FIGS. 2a and 2b has a large number of photosensitive pixels (e.g., 20/80 vision with a field of view of 10° corresponds to 18,000 pixels on a 3 mm chip) The cell stimulus can be a chemical, mechanical or electrical stimulus. In cases where electrical stimulation is employed, charge-balanced pulses are preferable, since such pulses are most suitable for chronic electrical stimulation. The retinal prostheses of FIGS. 2a and 2b can include electrical amplification in each pixel, which may be desirable to improve matching between optical pixel input intensity and the corresponding stimulus level. Preferably, the pixels of the retinal prosthesis are sized and spaced such that each pixel primarily stimulates one or a few retinal neural cells, in order to maximize spatial resolution. Further details on retinal prostheses suitable for use with the present invention are given in U.S. patent application Ser. No. 10/742,584, filed Dec. 19, 2003, and entitled “Interface for Making Spatially Resolved Electrical Contact to Neural Cells in a Biological Neural Network”.

When a retinal prosthesis as in FIGS. 2a and 2b is employed in a system as in FIG. 1, the optical pixel inputs are effectively controlled by image processor 106. This allows for a great deal of flexibility in system design, and in particular it allows for compensation of nonuniformity of response from pixel to pixel. The relation between each optical pixel input and the corresponding response of the stimulated neural cell can be quite complex. For example, this relation can depend on the type of neural cell being stimulated, the location of the electrode relative to the cell, and on the properties of the surrounding tissue. Various pixels in the retinal prosthesis may have different impedances (due to differences in tissue growth or electrode contamination) or different distances from the target cells and thus may require different pulse characteristics for cellular stimulation. These factors contribute to nonuniformity of the relation between the optical input and resulting stimulation in different areas of the implant. In addition, the retinal prosthetic system can at least partially provide intra-retinal signal processing (e.g., in cases where disease has degraded such processing), and in these cases the stimulus amplitude, duration, and other parameters can be a complex function of the optical image (contrast, average luminance, motion, etc.). Accordingly, the image processing provided by image processor 106 can also include processing to provide such functionality (e.g., temporal image processing).

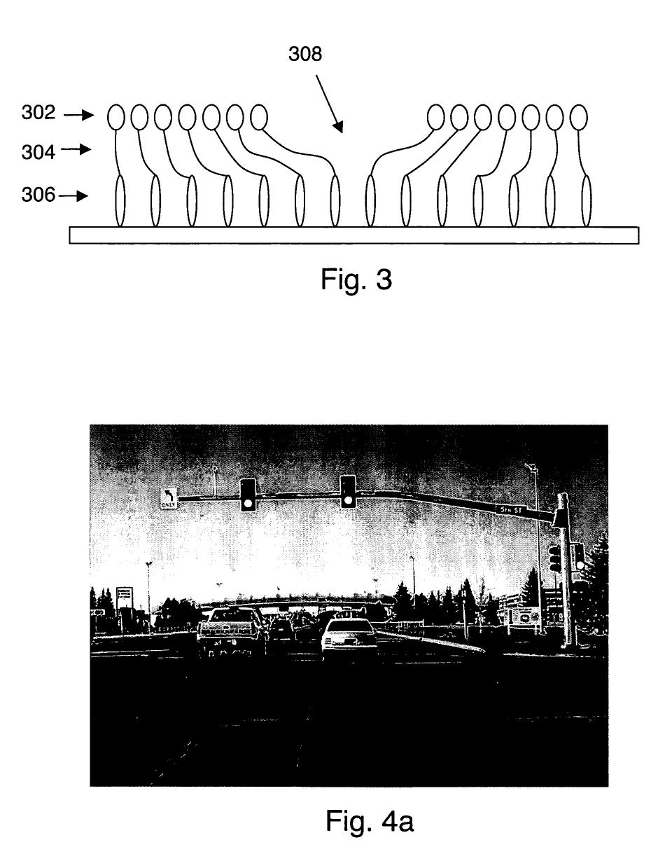

FIG. 3 shows a schematic diagram of the foveola of a human eye. Photoreceptors 306 are connected to neural cells 302 via nerve fibers 304. At the foveola 308, there is a region having photoreceptors 306, but no neural cells 302. Nerve fibers (Henle's fibers) connect foveolar photoreceptors 306 to neural cells 302 located away from the foveola. Typically these neural cells are about 0.5 mm away from the foveola. Thus, an image centered on the foveola will actually be processed neurally by bipolar cells and ganglion cells in a circular zone outside of the foveolar area. In order to most closely approximate normal vision, a retinal prosthesis system for use in this region of the retina preferably mimics these anatomical features. Such mimicry can be accomplished within the retinal prosthesis implant, by providing a non-local opto-neural mapping analogous to the natural non-local mapping shown on FIG. 3. However, this approach would add great complexity to the retinal implant.

A preferred approach for providing prosthetic vision in the foveolar region of the retina makes use of a retinal prosthesis having a localized relation between optical pixel input and neural stimulation (e.g., as shown on FIGS. 2a and 2b). The required non-local opto-neural mapping can be provided by position-dependent spatial image processing performed by image processor 106 on FIG. 1.

FIGS. 4a-c provide an example of what such position dependent spatial image processing would entail. FIG. 4a shows a normal visual scene (i.e., a typical optical image input 102 on FIG. 1). FIG. 4b shows the corresponding spatially processed image displayed by LCD 112 when the tracking subsystem has determined that a retinal prosthesis at the foveola is centered at the rightmost traffic light. The spatial image processing includes a radial scaling away from the center of the field of view, as required to provide the same effect as the natural anatomy shown on FIG. 3. There is also a region in the center of the field of view that is blank, corresponding to the absence of neural cells 302 in region 308 of FIG. 3. FIG. 4c is similar to FIG. 4b, except that the center of the field of view is at the car. Thus position-dependent spatial image processing is also a key feature of this aspect of the invention.

Note that the mapping between FIG. 4a and FIG. 4b (or FIG. 4c) also occurs in normal vision, due to the natural structure shown in FIG. 3. Thus in a prosthetic visual system according to the invention, position-dependent external spatial image processing can provide an equivalent mapping. Such external provision of the required mapping is clearly advantageous compared to approaches where this mapping is hard-wired into the retinal prosthesis (or implant), since greater flexibility is provided, and the implant is significantly simplified and less likely to require precise positioning during surgery and extensive customization for each individual patient. Since stimulation of retinal neural cells by a retinal prosthesis differs from natural retinal neural cell stimulation, some form of neural “learning” may be needed to achieve good vision, analogous to the learning used for modern cochlear implants. The flexibility provided by the present invention should advantageously facilitate such learning.

There are other cases where a non-local opto-neural map between optical image and neural stimulation can be desirable to compensate for partially lost retinal image processing functionality. For example, if “on” and “off” bipolar cells can be identified and separately stimulated, their stimulation level might be different from the corresponding optical pixel input intensity. In addition, there may be a need for temporal variation of the stimulation of neighboring pixels. For example, it may be useful to provide a time delay between stimulation pulses of different pixels.

The invention has been described above by way of example, so many modifications of the above examples also constitute practicing the present invention. For example, the invention can be applied to a single eye for monocular vision or to both eyes for binocular vision.

1. An external imaging unit located outside a mammalian eye having a photosensitive retinal prosthesis, the external imaging unit comprising:
an imager receiving an input optical image;
an image processor providing a processed image derived from said input optical image by image processing;
a display providing said processed image as an optical input to said eye, wherein light from said display is projected onto said retinal prosthesis by said eye; and
a tracking subsystem for determining a position of said retinal prosthesis relative to said display;
wherein said image processing includes spatial image processing dependent on said position of said retinal prosthesis relative to said display.

2. The external imaging unit of claim 1, wherein said processed image is displayed at an infrared wavelength.

3. The external imaging unit of claim 2, further comprising a screen substantially reflective at said infrared wavelength and substantially transparent to visible light, wherein light from said display is reflected from said screen into said eye, and visible light from an environment is transmitted through said screen into said eye.

4. The external imaging unit of claim 1, wherein said image processing further includes image intensification.

5. The external imaging unit of claim 1, wherein said image processing further includes temporal processing.

6. The external imaging unit of claim 1, wherein said spatial image processing includes one or more steps selected from the group consisting of rotating, translating, 1-D rectangular scaling, 2-D rectangular scaling, 2-D radial scaling, 2-D rectangular cropping and 2-D radial cropping.

7. The external imaging unit of claim 1, wherein said display provides a field of view substantially larger than a field of view of said retinal implant.

8. The external imaging unit of claim 7, wherein said display field of view is substantially equal to a field of view of said eye.

9. The external imaging unit of claim 1, wherein said display comprises a liquid crystal display having an array of pixels.

10. The external imaging unit of claim 9, wherein said display is selectively energized such that pixels having outputs projected at or near said retinal prosthesis are energized, and pixels having outputs projected away from said retinal prosthesis are not energized.

11. The external imaging unit of claim 1, wherein said display comprises an array of collimated light emitting diodes.

12. The external imaging unit of claim 11, wherein said array is selectively energized such that light emitting diodes having outputs projected at or near said retinal prosthesis are energized, and light emitting diodes having outputs projected away from said retinal prosthesis are not energized.

13. The external imaging unit of claim 12, wherein said energized light emitting diodes provide pulsed optical outputs.

14. A system for improving vision of a mammalian eye, the system comprising:
an imager located outside said eye and receiving an input optical image;
an image processor located outside said eye and providing a processed image derived from said input optical image by image processing;
a display located outside said eye providing said processed image as an optical input to said eye;
a photosensitive retinal prosthesis located inside said eye, wherein light from said display is projected onto said retinal prosthesis by said eye; and
a tracking subsystem for determining a position of said retinal prosthesis relative to said display;
wherein said image processing includes spatial image processing dependent on said position of said retinal prosthesis relative to said display.

15. The system of claim 14, wherein said spatial image processing provides an opto-neural mapping between said input optical image and retinal stimulation provided by said retinal prosthesis, wherein said opto-neural mapping is similar to a natural opto-neural mapping for said mammalian eye.

16. The system of claim 14, wherein said retinal prosthesis is positioned at a macula of said eye.

17. The system of claim 14, wherein said retinal prosthesis is disposed epiretinally or subretinally.

18. The system of claim 14, wherein said retinal prosthesis comprises discrete pixels, each pixel converting an optical pixel input to a localized electrical, mechanical or chemical neural cell stimulus in proximity to said optical pixel input.

19. The system of claim 18, further comprising an intra-ocular power supply providing electrical power to each of said pixels.

20. The system of claim 18, wherein said neural cell stimulus is a pulsed charge balanced electrical stimulus.

21. The system of claim 18, wherein said spatial processing compensates for pixel to pixel variation in a relation between said optical pixel input and said neural cell stimulus.

22. The system of claim 14, wherein said tracking subsystem includes an external photodetector array to detect radiation from reference points within said eye.

23. The system of claim 21, wherein said retinal prosthesis includes at least two tracking reference points.

24. The system of claim 23, wherein said reference points comprise corner cube reflectors.

25. The system of claim 23, wherein said reference points comprise light emitting diodes.

26. The system of claim 23, wherein said reference points comprise a fluorescent material.

27. The system of claim 14, wherein said retinal prosthesis includes an optical power receiver.

28. The system of claim 27, wherein a part of said display selected by said tracking subsystem emits light projected onto said optical power receiver for providing power to said retinal prosthesis.

29. A method for providing optical input to a mammalian eye having a photosensitive retinal prosthesis, the method comprising:
receiving an input optical image at a location outside said eye;
providing a processed image derived from said input optical image by image processing, wherein said image processing is performed at a location outside said eye; and
displaying said processed image with a display at a location outside said eye, wherein said displayed image is an optical input to said eye and light from said display is projected onto said retinal prosthesis by said eye;
wherein said image processing includes spatial image processing dependent on said position of said retinal prosthesis relative to said display.
